(19) United States
(12) Patent Application Publication
(10) Pub. No.: US 2020/0005924 A1
(43) Pub. Date: Jan. 2, 2020

(54) SYSTEM AND METHOD FOR MANAGING AND DISPENSING AN INHALABLE COMPOSITION, REGULATING ITS USE, AND COMMUNICATING DATA RELATED THERETO

(71) Applicant: Zorday IP, LLC, Mayfield Village, OH (US)

(72) Inventor: Brent M. Skoda, Mayfield Village, OH (US)

(73) Assignee: Zorday IP, LLC, Mayfield Village, OH (US)

(21) Appl. No.: 16/568,001

(22) Filed: Sep. 11, 2019

Related U.S. Application Data
(63) Continuation-in-part of application No. 16/375,372, filed on Apr. 4, 2019.
Application No. 16/375,372 is a continuation of application No. 15/047,332, filed on Feb. 18, 2016, now abandoned.
Application No. 15/047,332 is a continuation-in-part of application No. 16/386,600, filed on Apr. 17, 2019.
Application No. 16/386,600 is a continuation of application No. 15/048,575, filed on Feb. 19, 2016, now abandoned.
(60) Provisional application No. 62/118,341, filed on Feb. 19, 2015.
(60) Provisional application No. 62/118,869, filed on Feb. 20, 2015.

(51) Int. Cl. G16H 20/13; H04L 29/08

(52) U.S. Cl. CPC G16H 20/13; H04L 67/12

(57) ABSTRACT

A dispenser for dispensing an amount of liquid inhalable composition, the dispenser comprising: an optical radiation source, an optical detection sensor, an atomizer for atomizing the liquid inhalable composition, an event tracking electronic module for determining the number of vapes passing out of the dispenser; and an electronic visual display for displaying a vape count.

### CROSS-REFERENCE TO RELATED PATENT APPLICATIONS

This application is a continuation-in-part of, and claims priority to and the benefit of U.S. patent application Ser. No. 16/386,600, titled “SYSTEMS AND METHODS FOR INTELLIGENT VAPORIZERS,” and filed Apr. 17, 2019, which claims priority to and the benefit of U.S. patent application Ser. No. 15/048,575, titled “SYSTEMS AND METHODS FOR INTELLIGENT VAPORIZERS,” and filed Feb. 19, 2016, which claims priority to and the benefit of U.S. Provisional Patent Application No. 62/118,869, titled “SYSTEMS AND METHODS FOR INTELLIGENT VAPORIZERS,” and filed Feb. 20, 2015; this patent application is also a continuation-in-part of, and claims priority to and the benefit of U.S. patent application Ser. No. 16/375,372, titled “SYSTEMS AND METHODS FOR MEDICAL DISPENSING, MANAGEMENT AND MONITORING,” and filed Apr. 4, 2019, which claims priority to and the benefit of U.S. patent application Ser. No. 15/047,332, titled “SYSTEMS AND METHODS FOR MEDICAL DISPENSING, MANAGEMENT AND MONITORING,” and filed Feb. 18, 2016, which claims priority to U.S. Provisional Patent Application No. 62/118,341, titled “SYSTEMS AND METHODS FOR MEDICAL DISPENSING, MANAGEMENT AND MONITORING,” and filed Feb. 19, 2015. The contents of all of which are hereby incorporated herein by reference in its entirety for all purposes.

### SUMMARY OF THE INVENTION

This solution relates to: (i) a system and method of measuring and/or regulating a single user or multiple user's (Collectively, the “Users”) single inhalation (“Vape”) and/or cumulative inhalations (“Vapes”) through the use and application of wireless and/or mobile technology and/or the Internet, and the tracking of the geographic location of the Users vaporizer or vaporizers collectively and/or respectively; (ii) a system and method which immediately replenishes the Users supply of the product(s) (further explained below and referred to as an Inhalable Composition, Vaporizer, and/or Packaging Vehicle, collectively, “Product” or “Product(s)”).

This ensures that the Users always have an adequate supply of the Product(s) and do not run out of their supply of the Product(s), (“Automatically”), and/or replenished on a predetermined and scheduled, date, time, and/or geographic location (“Systematically”) which provides for a system that calls for replenishing a: vaporizer, an electronic cigarette in any form, a disposable vaporizer, a rechargeable vaporizer, or any product and/or delivery system capable of delivering a vaporizable substance (Collectively, the “Vaporizer”).

The system Automatically and/or Systematically replenishes a Product and/or Product(s) containing a vaporizable substance (i.e. which may include but is not limited to: a reusable cartridge, a refillable cartridge, a disposable cartridge, a box, a canister, a bottle, or any product capable of containing a vaporizable substance (Collectively, the “Packaging Vehicle”)); and/or Automatically and/or Systematically replenishing a vaporizable substance (“Inhalable Composition”) consumed by the Users, and, in particular, (iii) a system which functions to Systematically count and/or measure the Users Vapes per single inhalation (“Vape”), tracking the geographic location of the Users Vaporizer or Vaporizers collectively and/or respectively, monitors the remaining useful life of a power supply (i.e. battery) of a Vaporizer and/or Packaging Vehicle in the case that a particular Vaporizer and/or Packaging Vehicle is not rechargeable and requires replenishment collectively and/or respectively

The system Automatically reorders such an Inhalable Composition, Vaporizer, and/or Packaging Vehicle collectively and/or respectively when necessary, specified, and/or desired (further explained below), as well as a system and method whereby a Vaporizer can communicate the aforementioned data driven metrics with another or any other Vaporizer and vice versa or any grouping of Vaporizers, Packaging Vehicles, and/or Inhalable Compositions, through use and application of wireless and/or mobile technology and/or the Internet, together amongst the Users and/or directly from the Users to any relevant party comprising the supply chain known to one skilled in the art of the specific type Vaporizable Substance being delivered through the Vaporizers.

This could include but is not limited to: a medical professional, a doctor, a pharmacy entity, a pharmaceutical drug entity, a medical marijuana entity, a homeopathic drug entity, therapeutic drug entity, a supplement entity, an electronic cigarette entity, and/or known to one skilled in the art of Inhalable Compositions, Packaging Vehicles, for example, by way of illustration and not limitation, may include but is not limited to: a retailer entity, a re-distributor entity, a distributor entity, a wholesaler entity, a fulfillment entity, an exporter entity, an importer entity, a logistics entity, a manufacturing entity of the Vaporizer (“Primary Manufacturer of The Vaporizer”), and all supporting manufacturers, vendors, and/or affiliates whom supply the Primary Manufacturer of The Vaporizer, supply the manufacturing entity of the Inhalable Composition (“Primary Manufacturer of The Inhalable Composition”), supply the manufacturing entity of the Inhalable Composition (“Primary Manufacturer of The Packaging Vehicle”), and all supporting manufactures, vendors, and/or affiliates, whom supply the Primary Manufacturer of the Vaporizer, the Primary Manufacturer of The Inhalable Composition, and/or Primary Manufacturer of The Packaging Vehicle.

### BACKGROUND

In some embodiments, Users consume their supply of a Vaporizer(s), Packaging Vehicle(s), and/or Inhalable Composition(s), the Users may need to reorder a large quantity, rather than a smaller quantity which can be consumed within a reasonable time. A large quantity of the Vaporizer(s), Packaging Vehicle(s), and/or Inhalable Composition(s) may lose its effectiveness before the user is able to consume the supply.

In some embodiments, the User may need to be monitored by a Medical Doctor or other Overseer to ensure that the User's consumption of the Inhalable Composition stays below a pre-established threshold, or remains within acceptable limits.

In some embodiments, when the User does consume a supply of a Vaporizer(s), Packaging Vehicle(s), and/or Inhalable Composition(s), the present technology does not enable the Users to Automatically or Systematically replenish an adequate amount of their supply of a Vaporizer(s), Packaging Vehicle(s), and/or Inhalable Composition(s) which may vary from time to time based on variety of factors which may include but are not limited to: the Users usage, Users preferences; the composition, and/or the design and/or architecture of the Users Vaporizer(s), Packaging Vehicle(s), and/or Inhalable Composition(s).

Furthermore, because the present technology does not allow for the functions described in this Solution, it is inconvenient and unnecessary for the Users to manually place an order with a manufacturer, distributor, retailer, etc., when the Users need to acquire additional Vaporizer(s), Packaging Vehicle(s), and/or Inhalable Composition(s). Additionally, from time to time, Users may find their Products misplaced, lost, or stolen and need a means to locate their Products to avoid the time, expense, and inconvenience of reordering their Products.

Furthermore, the present technology does not allow for Users, their Products, as well as a system and method whereby a Users Products can communicate the aforementioned data driven metrics with another or any other Products and vice versa or any grouping of Vaporizers, Packaging Vehicles, and/or Inhalable Compositions, through use and application of a replenishment system in conjunction with wireless and/or mobile technology and/or the Internet, together amongst the Users and/or directly from a User to another party and/or entity, and any relevant party comprising the supply chain known to one skilled in the art of Vaporizers, Inhalable Compositions, and/or Packaging Vehicles.

For example, by way of illustration and not limitation, this may include: a retailer entity, a re-distributor entity, a distributor entity, a wholesaler entity, a fulfillment entity, an exporter entity, an importer entity, a logistics entity, a manufacturing entity of the Vaporizer (“Primary Manufacturer of The Vaporizer”), and all supporting manufacturers, vendors, and/or affiliates whom supply the Primary Manufacturer of The Vaporizer, supply the manufacturing entity of the Inhalable Composition (“Primary Manufacturer of The Inhalable Composition”), supply the manufacturing entity of the Inhalable Composition (“Primary Manufacturer of The Packaging Vehicle”), and all supporting manufactures, vendors, and/or affiliates, whom supply the Primary Manufacturer of the Vaporizer, the Primary Manufacturer of The Inhalable Composition, and/or Primary Manufacturer of The Packaging Vehicle.

What is needed is a system for Automatically and/or Systematically reordering a specified amount of the Vaporizer(s), Packaging Vehicle(s), and/or Inhalable Composition(s), while monitoring usage of the composition being consumed, where the time, quantity, Users preferences and desires, between orders may vary from time to time.

What is also needed is the ability for Users and/or and the relevant parties known to those skilled in the art of the Products whom comprise the supply chain of the Products to locate the Users Products, and efficiently and effectively manage their data together with themselves, their other Products, and the relevant parties known to those skilled in the art of the Products whom comprise the supply chain of the Products. For example, although Users may have many Products that may be similar in design, form, and/or function, it may be necessary to track to the respective and/or collective Usage to ensure the Users usage of the Products is within a measured and/or controlled manner, to ensure by monitoring that situations where the Users could potentially over use the Products and hurt themselves are anticipated and avoided.

### BRIEF DESCRIPTION OF THE DRAWINGS

FIG. 1 is a diagrammatical illustration of an inhalable composition monitoring and replenishment system, in accordance with the present invention;

FIG. 2 is a diagrammatical illustration of a first exemplary embodiment of an inhalable composition dispenser suitable for use in the composition monitoring and replenishment system of FIG. 1;

FIG. 3 is a diagrammatical illustration of a second exemplary embodiment of the inhalable composition dispenser of FIG. 2;

FIG. 4 is a flow diagram illustrating use of the inhalable composition dispenser of FIG. 2;

FIG. 5 is a diagrammatical illustration of a third exemplary embodiment of the inhalable composition dispenser of FIG. 2; and

FIG. 6 is a diagrammatical illustration of a fourth exemplary embodiment of the inhalable composition dispenser of FIG. 2.

### DETAILED DESCRIPTION

Certain administering devices use relatively small or varying amounts of the Inhalable Composition that need replenishment, and also may require replenishment at non-constant intervals, due to a variety of factors, which may include but are not limited to: the design, size, shape, voltage, Inhalable Composition capacity, which can respectively and/or collectively cause a user to consume the available Inhalable Composition at significantly different rates or time intervals, so a regular replenishment schedule is not practical.

The Inhalable Compositions disclosed herein could have a directed use of a certain number of Vapes (e.g., five vapes) per a specified time frame (e.g., per hour, per day, per week, per month, per year). If the User reaches the maximum threshold either collectively and/or respectively, which could be set by a Regulatory Body (e.g., Food and Drug Administration), a Medical Doctor, another User (i.e. a User's Parents, Parental Controls, a User's Coach, a User's Therapist, or a User's Psychologist), and/or any relevant entity composing the supply chain known to one skilled in the art of the Products.

It can be appreciated that facilities such as Airports, Employers, Regulatory Bodies, Educational Institutions, for example, will have the ability to notify Users through wireless technology, mobile technologies, and/or the Internet, if the User has violated certain rules pertaining to whether or not consumption of the Products is acceptable within their respective establishment(s), and/or offer advice to the Users as to where relevant to their present geographic permits the usage of the Products.

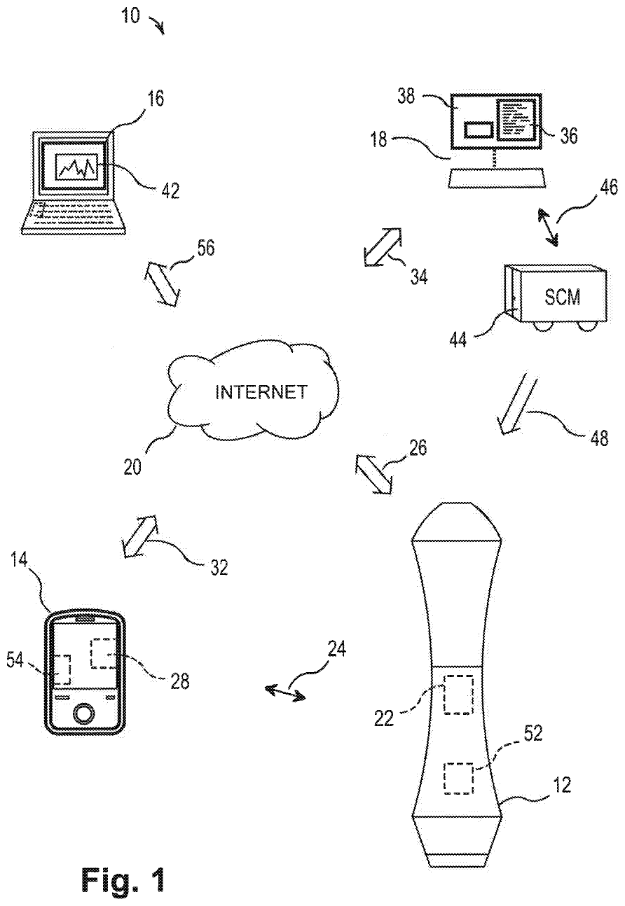

There is shown in FIG. 1 an Inhalable Composition monitoring and replenishment system 10 comprising an Inhalable Composition dispenser 12, a user personal communication device 14, a usage overseer computer terminal 16, and a supplier computer terminal 18, in accordance with an aspect of the present invention. The user may inhale or otherwise ingest, an Inhalable Composition from the Inhalable Composition dispenser 12, as is known in the relevant art.

The user composition monitoring and replenishment system 10 has a first function to insure that the supply status of the Inhalable Composition available to the user is monitored and automatically replenished as needed. When the Inhalable Composition dispenser 12 senses that the amount of useable Inhalable Composition remaining in the Inhalable Composition dispenser 12 is below a pre-specified threshold, as described in greater detail below, this determination may be wirelessly transmitted by a wireless transceiver 22 to the user personal communication device 14 using a short-range protocol 24, such as BLUETOOTH.

The user composition monitoring and replenishment system 10 has a second function to monitor the usage and consumption of the Inhalable Composition by the user. In the example shown, a medical doctor, a parent of the user, an athletic coach, a psychologist, or other responsible individual may use a graphic representation 42, or any other quantitative or qualitative indication, displayed on the usage overseer computer terminal 16 to determine the usage and cumulative consumption. As explained above, the graphic representation 42 can be used to determine the number of “Vapes” per a specified time frame for the User. If the User reaches the pre-specified maximum usage threshold, the usage overseer can respond accordingly. The usage overseer terminal 16 may be in communication with the user personal communication device 14 via a communication link 56 and the Internet 20. In an exemplary embodiment, the Inhalable Composition dispenser 12 may be in direct communication with the usage overseer computer terminal via a dispenser communication link 26 to the Internet 20.

It can be appreciated by one skilled in the relevant art that the user personal communication device 14 comprises a device which may include, but is not limited to any mobile form of mobile or wireless technologies, such as, for example, by way of illustration and not limitation, a WEBSITE, a MOBILE WEBSITE, a TELEPHONE, a CELLULAR PHONE, a SMART WATCH, a SMART PHONE; a computer within a motorized vehicle: such as a automobile, a truck, an airplane; a TABLET, a COMPUTER, a MOBILE APPLICATION, a GOOGLE TECHNOLOGY such as ANDROID DEVICE or a SIMILAR DEVICE, an APPLE TECHNOLOGY such as an iPHONE, iPAD APPLE TV or a SIMILAR DEVICE, a MICROSOFT TECHNOLOGY, a TELEVISION, or a DRONE.

In some embodiments, the Inhalable Composition dispenser 12 and/or the Users may communicate via the Internet 20 and/or wireless technology using wearable technologies such as, for example, by way of illustration and not limitation: an activity necklace, a wearable band or product incorporating a technology platform, a computerized chip, a microprocessor, and/or a communication device incorporating nanotechnology features, which may include but is not limited to: GOOGLE GLASSES or CONTACT LENSES incorporating the same, such as GOOGLE CONTACTS. The composition monitoring and replenishment system 10 has the ability to track the geographic location of the Products used by the user and supplied by the Products supplier that operates from the supplier computer terminal 18, among other communication sites.

In response to the transmittal from the wireless transceiver 22, a replenishment and monitoring app 28 resident in the user personal communication device 14 may function to access the Internet 20 via a user communication link 32 so as to initiate an Inhalable Composition purchase or acquisition for the user. The composition supplier terminal 18 may be accessed from the Internet 20 via a supplier communication link 34. The user request is represented in the illustration by a user purchase order or acquisition request 36 appearing on a transaction page 38 at the supplier computer terminal 18. Alternatively, the Inhalable Composition dispenser 12 may communicate directly with the composition supplier terminal 18 via the dispenser communication link 26 to the Internet 20.

In response to the acquisition request 36, the composition supplier terminal 18 may communicate with one or more supply chain members 44 via a wired or a wireless communication link 46 to initiate a shipment of the Inhalable composition via a ground, sea, or an air transport service 48. The shipment can be made to an address on file for the User, or may be made to the geographic location of the user as determined by a global positioning system (GPS) receiver 52 disposed in the Inhalable Composition dispenser 12 or by a similar GPS receiver 54 disposed in the user personal communication device 14. For certain commodities, such as a pharmaceutical product or a controlled substance, the user may be required to personally pick up and sign for the delivery. In such cases, the replenishment and monitoring app 28 may automatically provide a payment authorization to the acquisition request 36, such as authorizing a payment by a user credit or debit card.

It can be appreciated that supply chain members 44 utilize data to optimize the Users experience by monitoring which Inhalable Composition Products and/or Vaporizable Substances are being consumed by the user, and which are not. The supply chain members 44 can either automatically, or systematically, or by way of communication with the user, when the user initially purchases a variety of Products via the Supply Chain. The User then utilizes the variety of Products, and the replenishment and monitoring app 28 may determine whether the User enjoys a particular product, and that the user does not enjoy another Product and/or other Products.

This usage data is transmitted via wireless technology and/or the Internet from the User to the supply chain members 44, as described above. The supply chain members 44 receive and process the data, and then transmit a message back to the User via wireless technology and/or the Internet 20. The supply chain members 44 may inquire as to whether the user would like more or less of the Product and/or Products being consumed on a frequent basis. There may be additional inquires with the User as to whether the User would like more or less of the Product and/or Products being consumed on a less frequent basis. The User may then respond by transmitting preference(s).

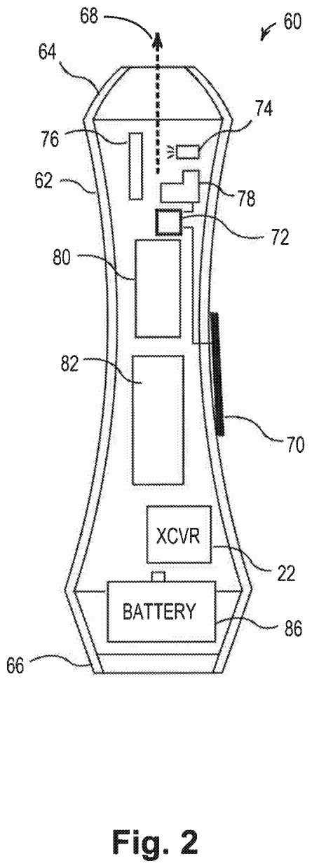

FIG. 2 shows a functional block diagram of an exemplary embodiment of an Inhalable Composition dispenser 60, in accordance with the present embodiment. Electrical and mechanical interconnections are not shown, for clarity of illustration. The Inhalable Composition dispenser 60 includes a housing 62, here shown in a cross-sectional view, enclosing the various components of the Inhalable Composition dispenser 60. The housing 62 is configured with a mouthpiece 64, and a base 66. The Inhalable Composition dispenser 60 may be used in the composition monitoring and replenishment system 10, in place of the Inhalable Composition dispenser 12, to provide user consumption information, both to the usage overseer terminal 16, as described above, as well as to an electronic visual display (EVD) 70 disposed on the housing 62 of the Inhalable Composition dispenser 60.

The electronic visual display 70 may comprise, for example, an LED screen, an LCD screen, or a touchscreen, and is provided to assist the user in tracking the amount of Inhalable Composition being consumed, and/or the cumulative amount of Inhalable Composition consumed over a specified period of time. Consumption of the Inhalable Composition is determined by counting the Vapes extracted from the Inhalable Composition dispenser 60 by the user. The electronic visual display 70 is powered and controlled by an EVD driver 72.

The Inhalable Composition dispenser 60 comprises an atomizer 80 and an Inhalable Composition cartridge 82, as shown in the diagram. The Inhalable Composition cartridge 82 is configured to hold and dispense the Inhalable Composition to the atomizer 80 for use by the user, or by the user's nominee. Atomized Inhalable Composition is produced by the atomizer 80, an action which is initiated or scheduled by the user or by the user's nominee. The Atomized Inhalable Composition is atomized to produce an intermittent Vape stream 68 that subsequently passes between: (i) an optical radiation source 74 such as, for example by way of illustration and not limitation, a light-emitting diode (LED), and (ii) an optical-detection sensor 76 such as, for example by way of illustration and not limitation, a photodiode. The optical radiation source 74, the atomizer 80, and the optical-detection sensor 76 are powered by a battery 86 during normal use of the Inhalable Composition dispenser 60.

When the atomizer 80 is triggered, the Inhalable Composition dispenser 60 is powered; and the optical-detection sensor 76 functions to continually sense optical radiation from the optical radiation source 74 that is incident upon the optical-detection sensor 76. As the user repeatedly inhales quantities of the intermittent Vape stream 68 from the Inhalable Composition dispenser 60, the intermittent Vape stream 68 acts to intermittently attenuate the amount of optical radiation from the optical radiation source 74 that reaches the optical-detection sensor 76. This change in light attenuation is continually monitored by the optical-detection sensor 76. This variation in optical radiation intensity upon the optical-detection sensor 76 is sensed and measured by an event tracking electronic module 78 that is in communication with the optical-detection sensor 76.

The event tracking electronic module 78 thus can provide a quantitative measurement of the cumulative amount of atomized Inhalable Composition delivered to the user, and the remaining amount of the Inhalable Composition available for use the by the user or by the user's nominee. This composition measurement can be used by the event tracking electronic module 78 to: (i) determine when an order or acquisition request 36 for Inhalable Composition should be placed to the composition supplier terminal 18 via the user personal communication device 14, (ii) measure and provide to the usage overseer terminal 16 a number of Vapes, for example, that the user has “puffed” or “vaped” via the event tracking module 78 and the user personal communication device 14; and/or (iii) provide an indication to the user as to whether or not the user's rate of consumption of the Inhalable Composition is within a recommended or acceptable range.

That is, the Inhalable Composition dispenser 60 functions to determine the supply status (i.e., the amount of Inhalable Composition remaining in the Inhalable Composition cartridge 82) from the cumulative amount of Inhalable Composition that has passed from the Inhalable Composition dispenser 60 as measured in the number of Vapes by those skilled in the art of personal vaporizers, taken by the user. This information may also be provided on the electronic visual display 70. The event tracking electronic module 78 may operate to “subtract” the cumulative amount of Inhalable Composition from the initial amount of Inhalable Composition in the Inhalable Composition cartridge 82 to calculate whether the supply status is deemed to have reached or fallen below a pre-specified threshold.

If the pre-specified threshold has been reached, or when data related to the usage level has been acquired, the wireless transceiver 22 may transmit this information to the user personal communication device 14, as shown in FIG. 1, from which: (i) a replacement order or acquisition request 36 may be initiated at the supplier computer terminal 18, in accordance with the procedure executed by the replenishment and monitoring app 28, and/or (ii) the usage overseer terminal 16 may be contacted in accordance with a pre-established procedure. In addition, the replenishment and monitoring app 28 may confirm receipt of the replenishment notification or acquisition request 36 from the wireless transceiver 22, and notify the user by an appropriate message on the display screen of the user personal communication device 14. If a personal pickup of a pharmaceutical or controlled substance is required, this may be also be indicated on the electronic visual display 70.

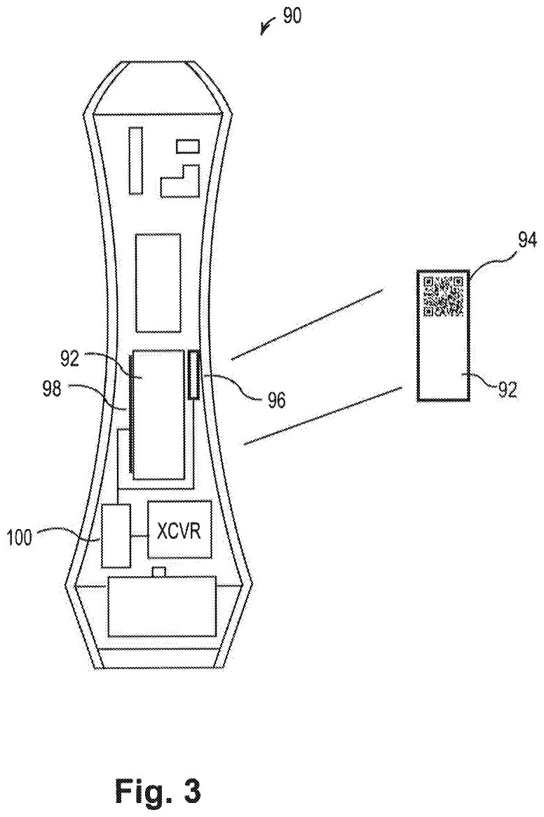

FIG. 3 shows a functional block diagram of an exemplary embodiment of an Inhalable Composition dispenser 90, in accordance with the present invention. The Inhalable Composition dispenser 90 may be used in the composition monitoring and replenishment system 10, in place of the Inhalable Composition dispenser 12, to provide a status of the amount of Inhalable Composition remaining in an Inhalable Composition cartridge 92. The Inhalable Composition dispenser 90 comprises a cartridge-type reader 96 which scans a Quick Response Code (QR code) 94, or alternatively embed a layer of rings or even scratches within the surface of the Inhalable Composition cartridge 92. In this configuration, a simple IR or RF reader (not shown) could be positioned inside, along the front end of the housing of the Inhalable Composition cartridge 92, in place of the cartridge-type reader 96. As the Inhalable Composition cartridge 92 is pushed/pulled in/out, each surface line will pass the IR or RF reader and thus give a unique ID (“IR/IF”). The benefit of this is that the IR or RF reader can be small, simple and inexpensive.

The QR code 94 on the Inhalable Composition cartridge 92 can also be used to determine the type of Inhalable Composition (i.e., e-liquid, medicine, flavor, etc.) contained in the Inhalable Composition cartridge 92. As may be appreciated, the properties or flavor of the e-liquid or Vaporizable Substance in the Inhalable Composition cartridge 92 may vary from one cartridge to another and identification by the QR code 94 provides a useful feature to the user.

In addition, the Inhalable Composition dispenser 90 may also comprise a fluid level sensor 98 to “read” the amount of e-liquid or Vaporizable Substance remaining in the Inhalable Composition cartridge 92. This may be accomplished by providing a “window” or translucent stripe (not shown) along the outer wall of the Inhalable Composition cartridge 92 through which the fluid level sensor 98 may determine the meniscus or level of the e-liquid or Vaporizable Substance remaining in the Inhalable Composition cartridge 92. This may also be accomplished by a User, overseer, and/or a third party who has preset the estimated or actual Vapes available per cartridge which will allow the Users and their Products to determine the meniscus or level of the e-liquid or Vaporizable Substance remaining in the Inhalable Composition cartridge 92.

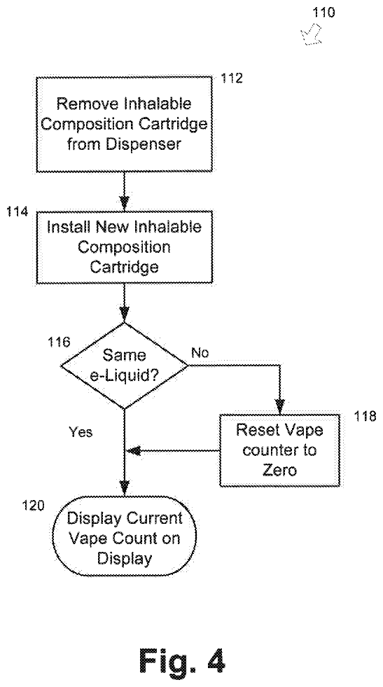

The inhalable composition dispenser 90 may function in accordance with a flow diagram 110, shown in FIG. 4. When the current Inhalable Composition cartridge 92 is empty, or when the user desires to replace the current Inhalable Composition cartridge 92 with another Inhalable Composition cartridge having a different flavor and/or type of Vaporizable Substance, for example, the current Inhalable Composition cartridge 92 is removed from the Inhalable Composition dispenser 90, at step 112.

After the replacement Inhalable Composition cartridge 92 is installed, at step 114, the cartridge sensing electronic module 100 reads the QR code 94, or other suitable indicator such as the IR/IF mentioned above, on the replacement Inhalable Composition cartridge 92 to determine whether the e-liquid or Vaporizable Substance in the replacement Inhalable Composition cartridge 82 is the same as the e-liquid or Vaporizable Substance in the removed Inhalable Composition cartridge 82, at decision block 116. If not, a Vape counter (not shown) in the event tracking electronic module 78, in FIG. 2, is reset, typically to “zero,” at step 118. This reset value becomes the current Vape count on the electronic visual display 70, at step 120. If, however, the e-liquid or Vaporizable Substance in the replacement Inhalable Composition cartridge 92 is the same as the e-liquid or Vaporizable Substance in the removed Inhalable Composition cartridge 92, the Vape counter is not reset, and the current Vape count continues to be displayed on the electronic visual display 70, at step 120.

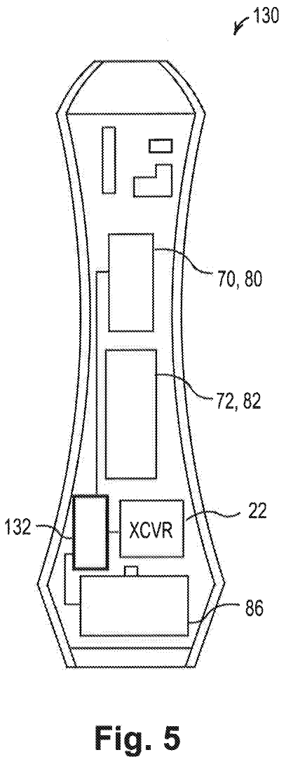

In an exemplary embodiment, shown in FIG. 5, an inhalable composition dispenser 130 may include a control module 132 that communicates with the user personal communication device 14 via the wireless transceiver 22. The replenishment and monitoring app 28 may set certain parameters for consumer usage of the inhalable composition products. For example, the usage overseer personnel using the usage overseer computer terminal 16 may recommend that the user be limited to administering the inhalable composition only three times per day, or a total of 15-30 Vapes per day. If the user attempts to exceed this amount, the replenishment and monitoring app 28 via Bluetooth, will instruct the inhalable composition dispenser 130 to suspend usage, until the following day if the consumers daily limit has been met. For such a contingency, the control module 132 may respond by terminating operating power from the battery or some from of power supply 86 to either or both of the cartridge 72, 82 and the atomizer 70, 80.

It can be appreciated that the GPS receivers 52, 54 will also help the user to not only locate the desired product and/or products but, in the event the user misplaces or has the product and/or products stolen, the GPS feature will also enable the manufacturer or a composition supplier to notice if the user is traveling for business, on vacation, etc. Also, the user may be notified when the automatic and/or systematic shipment of the product and/or products is ready to be sent to the user to replenish the product and/or products. Inquires with the user as to whether the shipment of the product and/or products should be rerouted to a current address, thus allowing the user to receive the product and/or products on time, and to personally sign for the delivery if the product is subject to such requirements.

In the case that the User Products, and/or another party, detect that the User Products collectively and/or respectively are not connected, and therefore that the User Products may have temporarily and/or permanently lost the Products connection collectively and/or respectively with another party, such as another Product, a medical professional, a pharmacy, or a regulatory body, the User Products can “disable themselves such that the Inhalable Composition dispenser terminates dispensing. It should be understood that communication among the User Product, a supplier, a medical professional, may be maintained via: (i) BLUETOOTH, (ii) a short range communication technology of any kind, (iii) a wired connection (e.g. a USB cable), or (iv) any other means necessary to communicate and/or regulate data and/or the Users Products collectively and/or respectively.

The Products can disable themselves, for any reason, by activating a controller which may disable the Products power supply once it detects that the Products collectively and/or respectively, have lost the Products connection. In an exemplary embodiment, such communication connection may employ wireless, cellular, wired, magnetic, for example, necessary to communicate among the Users and/or Users Products data (the “Critical Connection”) and one or more related entities. The related entities may include, but are not limited to: another Product, other Products, a medical professional, a regulatory body, a pharmacy, a group which includes the supply chain known to one skilled in the art of the industry or market where the Products being utilized for whatever use and application where the Products are being utilized.

The ability to manage the lost connection and control over the Products collectively and/or respectively and their associated power supply(s) can be programmed locally into a chip in the Products. In a “Direct End Method,” the chip may activate a controller to disable the power supply until the Critical Connection is reestablished.

Alternatively, or in conjunction with the Direct End Method, a “Remote End Method” includes the ability to manage the loss of the Critical Connection and control over the Products and their associated power supply and/or dispensing ability is collectively and/or respectively transmitted to the Users Products collectively and/or respectively at any time to the Users Products. This ability functions as long as the Critical Connection is valid by an entity which may include but is not limited to: another Product, others Products, a medical professional, a regulatory body, a pharmacy, an entity which comprises the supply chain known to one skilled in the art of the industry or market where the Products being utilized for whatever use and application where the Products are being utilized.

It can be appreciated that the reasons why the Direct End Method and the Remote End Method are important include, by way of illustration and not of limitation: (i) an implementation of certain embodiment may call for a medical professional and/or overseer to closely monitor a User's dosage at all times for number of reason which could include but are not limited to: a patient could be prescribed a controlled substance and if the Critical Connection is lost temporary and/or permanently cause pause for concern. Such reasons for concern include: an overseers suspicion of abuse, an overseer may have prescribed a medication that a User is allergic to may need to immediately suspend abuse, an overseer may have prescribed blood pressure medicine in conjunction with a wired and/or wireless blood pressure and/or hearth rate monitor which communicates with the Products and ultimately the overseer by way of the Products BLUETOOTH and/or another short range communication protocol and/or a wired and/or wireless technology and/or the Internet.

Another example may include a circumstance in which the Products are only so large and capable of storing certain data, because of the size of the hardware necessary is too large for the limited size of space inside of the Products, which could limit the amount of data that could be locally stored within the Products. For example, the entire pharmacopeia of FDA approved prescription drugs is significant in size and could not be stored locally within the products in its entirety due to the constraints of present technology.

Therefore, if a User has been prescribed a medication that does have a potentially harmful drug interaction with a large amount of other drugs prescription or otherwise, and could have a potentially harmful drug interaction with a large amount of other drugs prescription or otherwise, access to critical data would be needed. Clearly, the Critical Connection is necessary to ensure that all the critical data necessary is available and retrievable, to properly ensure that a harm drug interaction and/or potential abuse and/or prevention of any undesired outcome be it an allergic reaction or otherwise, is prevented from happening by harnessing the limited computing and data storage of the Products in conjunction with the Internet and massive database which contain the necessary data to ensure the integrity of the above described embodiments.

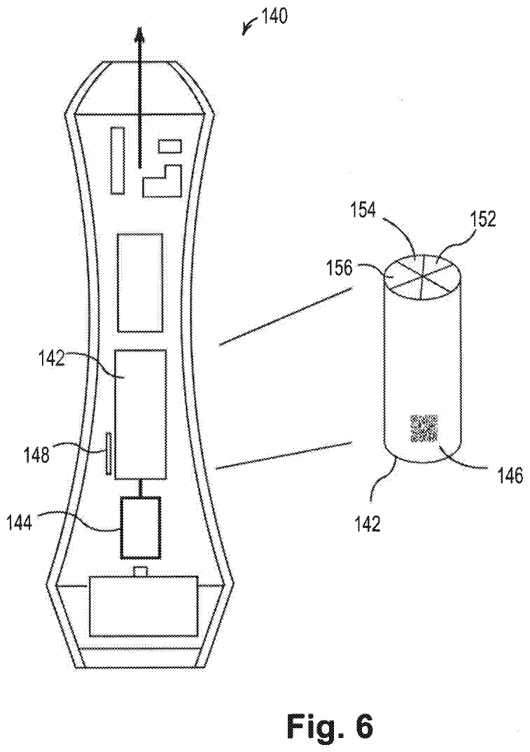

In an exemplary embodiment, shown in FIG. 6, an inhalable composition dispenser 140 may include a multi-chambered cartridge 142. The multi-chambered cartridge 142 includes a plurality of chambers, exemplified by chambers 152, 154, 156, where the number may be two or more chambers. Each chamber may contain a different medication or inhalable composition. The multi-chambered cartridge 142 may include one or more QR codes 146. A step motor 144 may be provided to rotate the multi-chambered cartridge 142 to a desired position to dispense a selected medication or inhalable composition. There may be a code reader 148 in the multi-chambered cartridge 142 to read the QR codes 146. The rotation and positioning of the multi-chambered cartridge 142 may be determined by the code reader 148 and the placements of the QR codes 146.

In an exemplary embodiment, a doctor may be overseeing a patient's health, and may have noticed that the patient needed more or less of a certain type of medication because he had several different types of ailments or had one ailment that required multiple medications in conjunction with each other. The doctor could, under such circumstances, adjust the medications taken by the patient, based on what the patient actually needed. For example, the patient may be wearing a glucose monitoring device, or some sort of mobile medical technology like a blood pressure monitor, and the device(s) communicated with the Inhalable Composition Dispenser via BLUETOOTH, cellular, a smart phone app and then ultimately the Internet.

The communication may be relayed to the doctor, who then reviews the patient's data, and has the ability to make adjustments in the dosage administered by the Inhalable Composition Dispenser. The Inhalable Composition Dispenser performs the vaporization of substances for the patient, based on actual needs, or levels of the vaporizable substance actually in your blood, administered by a mobile medical device, or by a non-mobile medical device, or by some combination thereof.

This would also provide the ability for the doctor to know if the patient actually took his medications as needed to avoid a potentially harmful outcome. This method of operation is very helpful from a business perspective for the doctor, and for the doctor's malpractice provider. The doctor may have the ability to detect that a patient is not responding correctly and can immediately increase, decrease, the patients vaporizable substance and respective dosages, as needed.

For a cancer patient, this method of operation could be extremely helpful when the cancer patient is using several experimental types of vaporizable substances. If the doctor noticed that the cancer patient was not responding particularly well to a given vaporizable substance, the doctor can then immediately respond accordingly by providing more of the vaporizable substance that is working and effective. In some cases, this action could possibly either save the patient's life, or significantly extend the patient's life when apparent that a certain vaporizable substance (i.e. medication) was effective, when through traditional routes and the present technology, an effective vaporizable substance may never have been discovered during the patient's life time.

Further, an overseer would have the ability to manually review the patient's medication data, device usage, monitor effectiveness, etc. Alternatively, this could also be performed by some form of predictive technology and/or a published medical database which contained instructions for doctors regarding what to prescribe to their patients once certain medical guidelines were met. This information would also include what vaporizable substances, substance dosages, and variations to prescribe to be most effective.

This data could be relayed back to the patient's Inhalable Composition Dispenser, and/or if the patient's cartridge does not contain the vaporizable substances (i.e. medications) the patient's needs, based on his previous medications effectiveness or non-effectiveness, the appropriate cartridge containing the appropriate vaporizable substances could be automatically and/or systematically issued and/or shipped to the patient. This action maximizes the medication's effectiveness and minimizes the patient's pain and suffering.

As can be appreciated by one skilled in the art, a small quantity of a liquid medication “goes a long way.” For example, a 0.45 ml quantity can provide approximately 100 to 120 vapes or puffs. The chambers in the disposable cartridges are not particularly large, but are effective and provide for enough uses before the patient needs to dispose of the empty cartridge and obtain a new cartridge.

The disclosed system includes the ability to remotely switch the functions of the Inhalable Composition Dispenser and Products between the ability for the Inhalable Composition Dispenser and/or cartridge and/products to either dispense a powder, vaporize anything that can be vaporized, or dispense a liquid. This is valuable because some products are more effective to vaporize, whether it be herbal, liquids, etc. Some are better to drink, some are better to ingest in a powder like form, and different cartridge could contain different vaporizable substances and or other substances.

When the cartridge is installed on the device either through a database stored on a mobile app, for example, or via a database located on a server accessed via the Internet and/or BLUETOOTH and/or some kind local short range transmission protocol and/or cellular technology, etc., within the cartridge, ID's located on the male and female connection between the cartridge could tell the device whether to dispense liquid, vaporize the substance, or dispense a powder form substance, based on preset instructions located in a database which we may update from time to time as formulas improve and change and be best delivered through one type delivery or another.

A user may also have the ability to manage and between, and switch modes of, the products through a smartphone application. Thus, if the user wanted change from the feature to vaporize, dispense liquid, or dispense a power, or possibly a small capsule or pill, that can be done.

The ability to adjust voltage remotely may also be done on the device itself. This feature may be provided as some substances require different voltages to properly vaporize, dispense, etc. The voltage is controlled by firmware. A number in a register corresponds to a specific output voltage. The program can change that number any time it likes. For instance the user could read the cartridge chip, or pass the voltage value from an external source to the program via BLUETOOTH. The proper voltage may be found from a table using the cartridge fluid ID, or the cartridge can store the voltage value itself. So the program is preferably running in firmware, and controlling the voltage. The program listens to the GPS and Bluetooth, reads the cartridge chip, and updates the display.

As can be appreciated by one skilled in the art, the above-disclosed solutions comprise: (i) a method and system to automatically and systematically notify a usage overseer of the user consumption and user rate of consumption of Inhalable Composition; (ii) a method and system to automatically and systematically replenish the Products and/or inhalable composition dispensers, collectively and/or respectively; (iii) a method and system to count and/or measure the User's Vapes; (iv) a system and method to track the geographic location of the Products, together with the Internet, and/or wireless technologies (e.g., smart watch, smart phone, wearable technology, drones, mobile app, website, etc.), and/or another Product together with the Users, respectively and/or collectively; (v) a system and method to allow for the ability to communicate of all the relevant data amongst the Users collectively and/respectively, the Products collectively and/or respectively, the inhalable composition dispensers collectively and/or respectively; and, (vi) a system and method to communicate this data with the manufacturers and any other parties that may comprise the relevant supply chain or otherwise. Operation may be enabled by means of wireless, Wi-Fi and/or Bluetooth technologies.

It is to be understood that the description herein is only exemplary of the solution, and is intended to provide an overview for the understanding of the nature and character of the disclosed systems for dispensing Inhalable Compositions. The system may further function in accordance with the present solution by using an Inhalable Composition dispenser having: an Inhalable Composition level sensor, an optical radiation source, and a light-detecting device, so as to be able to both sense fluid level remaining in the Inhalable Composition cartridge, and to monitor the cumulative amount of Inhalable Composition measured as the number of Puffs or Vapes taken by a user, as described above. The accompanying drawings are included to provide a further understanding of various features and embodiments of the method and devices of the solution which, together with their description serve to explain the principles and operation of the solution.

What is claimed is:

1. A dispenser configured to dispense and tack an amount of composition inhaled, the dispenser comprising:
a housing;
an optical radiation source disposed within the housing;
an optical detection sensor disposed within the housing and configured to detect radiation from said optical radiation source;
an atomizer disposed within the housing, the atomizer configured to atomize a composition into vapes inhalable by a user of the dispenser, wherein the atomizer is positioned to direct atomized composition between the optical radiation source and the optical detection sensor;
an event tracking electronic module in communication with the optical detection sensor for determining a number of vapes passing between the optical radiation source and the optical detection sensor; and
an electronic visual display in communication with the event tracking electronic module for displaying the number of vapes.

2. The dispenser of claim 1, wherein the composition comprises one of a liquid, powder, medicine or a flavor.

3. The dispenser of claim 1, wherein a vape is a single inhalation of the atomized composition.

4. The dispenser of claim 1, wherein the event tracking electronic module is configured to determine the number of vapes per a specified time period.

5. The dispenser of claim 1, wherein the event tracking electronic module is configured to determine a cumulative amount of atomized composition delivered to the user.

6. The dispenser of claim 1, wherein the event tracking electronic module is configured to determine a remaining amount of composition available to be atomized.

7. The dispenser of claim 1, wherein the event tracking electronic module is configured to determine that a reorder of composition is to be placed.

8. The dispenser of claim 7, wherein the event tracking electronic module is configured to place the order via a mobile device of the user.

9. The dispenser of claim 1, wherein the optical radiation source comprises a light emitting diode.

10. The dispenser of claim 1, wherein the optical detection sensor comprises a photodiode.

11. A dispenser comprising:
a cartridge comprising a vaporizable substance;
a sensor for detecting an amount of vaporizable substance in the cartridge;
a reader is configured to scan the cartridge to identify a type of vaporizable sub stance;
an atomizer for atomizing the vaporizable substance into vapes inhalable by a user of the dispenser;
a wireless transceiver;
a cartridge sensing electronics module is configured to communicate via the wireless transceiver, responsive to the sensor, a supply status of the type of vaporizable sub stance.

12. The dispenser of claim 11, wherein the supply status is communicated to a personal communication device.

13. The dispenser of claim 11, wherein the supply status is communicated to a supplier terminal via a dispenser communication link.

14. The dispenser of claim 11, wherein the supply status identifies a remaining amount of the type of vaporizable substance.

15. The dispenser of claim 11, further comprising a global positioning system (GPS) and wherein cartridge sensing electronics module is configured to communicate via the wireless transceiver GPS information.

16. The dispenser of claim 11, wherein the reader is configured to scan a QR code.

17. The dispenser of claim 11, wherein the cartridge is a multi-chamber cartridge.

18. The dispenser of claim 17, wherein the readier is further configured to determine a rotation and position of the multi-chamber cartridge.

19. The dispenser of claim 17, further comprising a motor for rotatably positioning the multi-chamber cartridge.

20. The dispenser of claim 11, further comprising a vape counter to count vapes based on the type of vaporizable substance in the cartridge.
